(19) United States
(12) Patent Application Publication
(10) Pub. No.: US 2018/0368727 A1
(43) Pub. Date: Dec. 27, 2018

(54) APPARATUS FOR TOPICAL APPLICATION OF MATERIAL

(71) Applicant: CUTITRONICS LTD, East Kilbride (GB)

(72) Inventors: David HEATH, East Kilbride (GB); Barry HOCHFIELD, East Kilbride (GB)

(21) Appl. No.: 16/065,005

(22) PCT Filed: Dec. 23, 2016
(86) PCT No.: PCT/GB2016/054075

§ 371 Date: Jun. 21, 2018

(30) Foreign Application Priority Data
Dec. 23, 2015 (GB) 1522851.3

(51) Int. Cl. A61B 5/053; A61B 5/01; A61N 1/04; A61N 1/32; A61N 1/30

(52) U.S. Cl. CPC A61B 5/0531; A61B 5/01; A61B 5/0008; A61N 1/325; A61N 1/303; A61N 1/0428

(57) ABSTRACT

The present invention relates to apparatus for topical application of material for cosmetic or medical purposes. The apparatus comprises measurement apparatus (154), (156), (158), (152) configured to measure a property of skin of a human or animal 5 subject,actuating apparatus (146), (148), (152) configured to change a property of the skin of the human or animal subject and application apparatus (162) configured to apply material for cosmetic or medical purposes to the skin. The apparatus further comprises a processor (132) which is configured: to receive operational data based on data received from each of plural other apparatus for topical application of material, (10) said received operational data pertaining to operation of the other apparatus for topical application of material; and to control the apparatus for topical application of material in dependence on the property measured by the measurement apparatus and the operational data.

### FIELD OF THE INVENTION

The present invention relates to apparatus for topical application of material for cosmetic or medical purposes. The present invention also relates to an arrangement for topical application of material for cosmetic or medical purposes comprising plural such apparatus for topical application of material for cosmetic or medical purposes. The present invention further relates to a method of topically applying material for cosmetic or medical purposes.

### BACKGROUND ART

Apparatus which measure properties of the skin and apply cosmetics are known. For example US 2009/0025747 discloses an approach which involves determining reflectance or texture attributes of skin by optical techniques and then applying a reflectance modifying agent, such as a pigmented cosmetic material, to the skin in dependence on analysis of the attribute data.

The present inventors have recognised known approaches, such as the approach of US 2009/0025747, to have shortcomings.

The present invention has been devised in light of the inventors' appreciation of such shortcomings. It is therefore an object for the present invention to provide improved apparatus for topical application of material for cosmetic or medical purposes. It is a further object for the present invention to provide an arrangement for topical application of material for cosmetic or medical purposes comprising plural such improved apparatus for topical application of material for cosmetic or medical purposes. It is a yet further object for the present invention to provide an improved method of topically applying material for cosmetic or medical purposes.

### STATEMENT OF INVENTION

According to a first aspect of the present invention there is provided apparatus for topical application of material for cosmetic or medical purposes, the apparatus comprising:
measurement apparatus configured to measure a property of skin of a human or animal subject;
actuating apparatus configured to change a property of the skin of the human or animal subject;
application apparatus configured to apply material for cosmetic or medical purposes to the skin; and
a processor which is configured: to receive operational data based on data received from each of plural other apparatus for topical application of material, said received data pertaining to the operation of the other apparatus for topical application of material; and to control the apparatus for topical application of material in dependence on the property measured by the measurement apparatus and the operational data.

Apparatus according to the present invention is for topical application of material for cosmetic or medical purposes. The apparatus comprises measurement apparatus configured to measure a property of skin of a human or animal subject and actuating apparatus configured to change a property of the skin of the human or animal subject. The apparatus also comprises application apparatus configured to apply material for cosmetic or medical purposes to the skin. The apparatus further comprises a processor which is configured to receive operational data based on data received from each of plural other apparatus for topical application of material, said received data pertaining to the operation of the other apparatus for topical application of material.

The operational data may, for example, be received from a central database which stores on an ongoing basis data received from plural other apparatus for topical application of material. The received data may have been processed on a central server, which may comprise the central database, to provide plural profiles, such as Italian women between age forty and forty-nine, British women between age thirty and thirty-nine, etc. One of the plural profiles which is appropriate to the user of the present apparatus for topical application of material may be received in the apparatus for topical application of material. The operational data may therefore comprise at least one profile. The processor is also configured to control the apparatus for topical application of material in dependence on the property measured by the measurement apparatus and the operational data. Control of the apparatus for topical application of material is therefore informed by measurements made by the apparatus for topical application of material itself and also by earlier received data pertaining to operation of other apparatus for topical application of material. The present inventors have become appreciative that control may be improved when based not only on local measurements, i.e. measurements made with the measurement apparatus, but also on data pertaining to operation of other apparatus for topical application of material.

The apparatus for topical application of material may be configured such that at least one of the actuating apparatus and the application apparatus is controlled in dependence on the property measured by the measurement apparatus and the operational data. Control of at least one of the actuating apparatus and the application apparatus therefore may be informed by measurements made by the apparatus for topical application of material itself and also by earlier received data pertaining to operation of other apparatus for topical application of material.

Alternatively or in addition, the apparatus for topical application of material may comprise a user interface, the apparatus for topical application of material being configured to inform the user by way of the user interface in dependence on the property measured by the measurement apparatus and the operational data. The user interface may comprise a display, the apparatus for topical application of material being configured to inform the user by way of the display. For example, in dependence on the property measured by the measurement apparatus and the operational data, the processor may be operative to determine that a formulation of the material for cosmetic or medical purposes should be changed and to inform the user accordingly by way of the display. More specifically, a changed formulation may be sent to the user, for example in the form of a replacement cartridge containing the changed formulation, without user input and the user may be informed accordingly. According to another example, the processor may be operative to make a determination concerning use of the apparatus for topical application of material, such as in respect of timing and frequency of use, volume of material for cosmetic or medical purposes to be applied or rate of application of material for cosmetic or medical purposes, and to inform the user accordingly. Alternatively or in addition, the user interface may comprise a communication port. In use, the communication port may provide for communication of data to further apparatus, such as a Personal Computer. According to one of the previous examples, the processor may be operative to convey by way of the communication port data corresponding to a determination that a formulation of the material for cosmetic or medical purposes should be changed. In use, data conveyed by way of the communication port may be received by the Personal Computer which then may be operative to provide the data in a form perceptible by the user. Alternatively or in addition, the user interface may comprise a loudspeaker by way of which the user is informed.

The apparatus for topical application of material for cosmetic or medical purposes may further comprise data communication apparatus which is operative to receive the operational data. The operational data may be received from a remote data store. The data communication apparatus may be further operative to transmit data pertaining to the operation of the apparatus for topical application of material to a remote data store. More specifically the data communication apparatus may be operative to transmit data relating to operation of at least one of the measurement apparatus, the actuating apparatus and the application apparatus. For example, the transmitted data may be based on and more specifically may comprise at least one of: measurements made with the measurement apparatus; characteristics of an actuating signal received by the actuating apparatus; and characteristics of an actuating signal received by the application apparatus. Alternatively or in addition, the data communication apparatus may be operative to transmit data relating to measurement made by way of at least one sensor comprised in the apparatus for topical application of material. More specifically the apparatus for topical application of material may comprise at least one of a temperature sensor and a humidity sensor. The transmitted data may therefore relate to measurement made by at least one of a temperature sensor and a humidity sensor. Alternatively or in addition, the apparatus for topical application of material may be operative to determine its location and to provide corresponding location data, for example location data as provided by location determining apparatus, such as a GPS receiver, comprised in the apparatus for topical application of material. The apparatus for topical application of material may be operative to transmit the location data by way of the data communication apparatus. The operational data may be based on corresponding data from other apparatus for topical application of material, such as data relating to operation of at least one of the measurement apparatus, the actuating apparatus and the application apparatus of the other apparatus, measurement data from the other apparatus and location data from the other apparatus.

Where the apparatus for topical application of material comprises at least one of a sensor and location determining apparatus, the processor may be configured to further control the apparatus for topical application of material in dependence on at least one of measurement data from the sensor and location data from the location determining apparatus. More specifically the processor may be operative to effect control in dependence on the operational data having regard to at least one of measurement data from the sensor and location data from the location determining apparatus. For example, the processor may be operative to make an inference regarding climate based on the location data and to control the apparatus for topical application of material in dependence on operational data selected in accordance with climate. By way of further example, the processor may be operative to control the apparatus for topical application of material in dependence on the operational data having regard to local temperature and humidity measurements.

According to a second aspect of the present invention there is provided an arrangement for topical application of material for cosmetic or medical purposes, the arrangement comprising: plural apparatus for topical application of material for cosmetic or medical purposes, each according to the first aspect of the present invention; and a data store remote from the plural apparatus. The data store may be comprised in a computer server arrangement. The data store may be comprised in a cloud based arrangement.

Each of the plural apparatus for topical application of material may be operative to receive operational data from the data store. Alternatively or in addition, each of the plural apparatus for topical application of material may be operative to transmit data pertaining to the operation of the apparatus for topical application of material to the data store. The utility of the present invention may increase with the number of plural apparatus for topical application of material. Data from a larger pool of apparatus for topical application of material may provide for improved operational data. The operational data which the data store provides to apparatus for topical application of material may therefore be updated on an ongoing basis in dependence on fresh data received from the plural apparatus for topical application of material.

Each of the plural apparatus for topical application of material may therefore be operative on the basis of two feedback loops. A first feedback loop may be local to the apparatus for topical application of material and may comprise the measurement apparatus comprised in the apparatus and at least one of the actuation apparatus and the application apparatus comprised in the apparatus. A second feedback loop may be remote in part from the apparatus for topical application of material and may comprise the plural apparatus for topical application of material, the data store and each of the plural apparatus for topical application of material. One of the first and second feedback loops may influence the other feedback loop. More specifically, the second feedback loop may be operative to influence the first feedback loop. The processor comprised in apparatus for topical application of material may be operative to change how the apparatus for topical application of material and more specifically at least one of the actuation apparatus and the application apparatus is controlled in dependence on the operational data. The operational data may therefore provide for improved control of at least one of the actuation apparatus and the application apparatus.

The data store may be operative to form at least one profile in dependence on the data received from each of plural other apparatus for topical application of material. Control of at least one of the actuating apparatus and the application apparatus may therefore be in dependence on local measurements, i.e. measurements made with the measurement apparatus, and a profile received from the data store. The at least one profile formed by the data store may be modified in dependence on further data received from each of plural other apparatus for topical application of material. The at least one profile may thus be refined or modified on an ongoing basis.

A profile may be formed in respect of a predetermined category of human or animal subject. The remote data store may therefore store plural different profiles, one of the plural profiles being selected and transmitted to apparatus for topical application of material for cosmetic or medical purposes in accordance with a characteristic of the human or animal subject with which the apparatus for topical application of material for cosmetic or medical purposes is to be or is being used.

The arrangement for topical application of material may be configured to analyse the data received from the plural other apparatus for topical application of material. The analysis may be by way of a central processor remote from the plural other apparatus for topical application of material. The central processor may, for example, be comprised in the same apparatus as the data store. Analysis may comprise determining at least one characteristic common to data received from the plural other apparatus for topical application of material. At least one profile may be formed in dependence on the analysis. Following receipt of data from plural other apparatus for topical application of material and analysis thereof, further data may be received from further apparatus for topical application of material. The further data may be analysed in light of the previous analysis. If the further data is determined to be inappropriate for the at least one profile, the arrangement for topical application of material may be operative to form a fresh profile in dependence on the determination.

Further embodiments of the second aspect of the present invention may comprise one or more features of the first aspect of the present invention and vice-versa.

As mentioned above, the apparatus for topical application of material comprises measurement apparatus which is configured to measure a property of skin of a human or animal subject. More specifically the measurement apparatus may be configured to measure an electrical property of skin of a human or animal subject.

The measurement apparatus may be configured to measure an impedance of the skin. Apparatus according to the present invention may be operative to make a determination in respect of permeability of the measured skin in dependence on the measured impedance. The apparatus may be operative to make the determination in dependence on a model which relates measured impedance and permeability to each other. The measured property of the skin may therefore comprise impedance and more specifically a real part of impedance. The measurement apparatus may comprise spaced apart measurement members and a signal generator, the signal generator being operative to apply a signal to the spaced apart measurement members.

As mentioned above, the apparatus for topical application of material comprises actuating apparatus which is configured to change a property of the skin of the human or animal subject. More specifically the actuating apparatus may be configured to change a permeability of the skin of the human or animal subject. The actuating apparatus may be operative in dependence on the measured property of the skin. Alternatively or in addition, the actuating apparatus may be operative in dependence on the data received based on plural measurements. The measured property or the data received, for example, may be such that it is determined that an increase in permeability is desirable with the actuating apparatus being operative accordingly to increase the permeability of the skin.

The actuating apparatus may be configured to change a permeability of the skin by application of an electric signal to the skin. The electric signal may be at least one of substantially constant and varying, such as at least one pulse. The actuating apparatus may be configured to change a permeability of the skin by application to the skin of at least one of: an electric potential signal; and an electric current signal. As described further below, the electric potential signal and the electric current signal may be applied to the skin at one of: different times; and substantially a same time. In certain forms, the electric potential signal may be applied to create pathways and then the electric current signal may be applied to maintain the created pathways. Therefore the actuating apparatus may be configured to apply the electric potential signal and then to apply the electric current signal.

The actuating apparatus may comprise spaced apart members which are susceptible of carrying current and which are operative to develop an electric potential therebetween. Each spaced apart member may have the form of an electrode. The actuating apparatus may be configured such that the spaced apart members are electrically isolated from the skin when the actuating apparatus bears against the skin. The actuating apparatus may be configured to change a level of applied electric potential. The level of applied electric potential may be changed, for example, to take account of different skin thicknesses and types. Alternatively the level of applied electric potential may be changed in dependence on at least one of measurement of a property of the skin and the received data based on plural measurements.

The actuating apparatus may be configured to apply the electric potential signal to the skin as a pulse. The actuating apparatus may be configured to change at least one of duration of the applied pulse and a time period between applied pulses. The duration of the applied pulse may be changed, for example, to take account of different skin thicknesses and types. The time period between applied pulses may be changed, for example, to take account of different skin thicknesses and types.

Alternatively at least one of the duration of an applied pulse and the time period between applied pulses may be changed in dependence on at least one of measurement of a property of the skin and the received data based on plural measurements. The duration of the pulse, the time period between applied pulses and perhaps also the level of the pulse as described above may be determined to provide a desired effect, such as in respect of a diameter of pathways formed in the skin or density of pathways formed in the skin.

Alternatively or in addition the actuating apparatus may be configured to apply an electric current signal to the skin. The application of an electric current to the skin has been found to increase permeability of the skin. The actuating apparatus may comprise spaced apart current applying members which carry current and which are operative to pass current therebetween by way of the skin when the spaced apart current applying members are in contact with the skin. The actuating apparatus may be configured such that the electric current is substantially constant while it is being applied to the skin. Each spaced apart current applying member may have the form of an electrode. Each electrode may lack the insulating layer described above with reference to electric potential application to thereby provide a conductive path between the electrode and the skin.

Application of electric current to the skin typically reduces the impedance of the skin with the impedance being a function of duration of application of the electric current and density of the electric current. Decreased skin impedance normally reflects increased permeability of the skin on account of changes to the skin caused by the flow of electric current. The increase in permeability and reduction in impedance is understood to be because of recruitment of appendageal pathways as transport pathways. This phenomenon is normally termed iontophoresis. Current flowing during iontophoresis is termed iontophoretic current. In contrast with application of electric potential, application of electric current provides for movement of the material for cosmetic or medical purposes along the pathways.

In certain embodiments, the application of an electric potential and an electric current to the skin may be used together in the apparatus to advantageous effect. An electric potential may be applied to the skin to create pathways therein and then application of the electric potential may cease while an electric current is applied to the skin to maintain the pathways created by the electric potential. The actuating apparatus may be configured accordingly. More specifically the actuating apparatus may comprise spaced apart members which are operative to apply the electric potential and spaced apart current applying members which are operative to apply the electric current.

As mentioned above, the application apparatus is configured to apply material for cosmetic or medical purposes to the skin. More specifically the application apparatus may be configured to apply a cosmetic to the skin. The apparatus for topical application of material for cosmetic or medical purposes may therefore be apparatus for topical application of material for cosmetic purposes alone of cosmetic and medical purposes. Furthermore the apparatus may be configured for topical application of material for cosmetic purposes solely of cosmetic and medical purposes. The material for cosmetic purposes may be of a particular constitution which is intended to effect an improvement such as in respect of the cosmetic appearance of the like of the skin of the face. The material therefore may be a substance. The material for cosmetic purposes may be a cosmetic agent. The material for cosmetic purposes may comprise at least one of primer, concealer, foundation, bronzer, setting spray, cleanser, toner, skin-care lotion, moisturiser, humectant, sunscreen, tanning oil, tanning lotion, skin lightener and exfoliant.

The application apparatus may be operative in a changing fashion in dependence on the operational data and measurements made by the measurement apparatus. The application apparatus may be operative in dependence on an actuating signal received by the actuating apparatus, such as an actuating signal generated by the processor. The actuating signal may be generated by the processor in dependence on the operational data and measurements made by the measurement apparatus. The application apparatus may be operative to change at least one of: a quantity of material applied to the skin; a rate of application of material to the skin; and a consistency of material applied to the skin. Alternatively or in addition the application apparatus may be operative to change a composition of material applied to the skin in dependence on the operational data and measurements made by the measurement apparatus.

The application apparatus may comprise a material actuator which is operative to dispense material for cosmetic or medical purposes from the apparatus for topical application of material. The material actuator may comprise a pump. The application apparatus may comprise a reservoir which is configured to hold material for cosmetic or medical purposes. The material actuator may be operative to dispense material for cosmetic or medical purposes from the reservoir. The application apparatus may comprise at least one dispensing aperture through which material for cosmetic or medical purposes is dispensed from the apparatus for topical application of material to the skin of the subject. The at least one dispensing aperture may be disposed adjacent to at least one of the measurement apparatus and the actuating apparatus.

The material may be fluent material. The material may be fluid and more specifically a liquid albeit perhaps a viscous liquid such as a cream, paste, gel or foam.

The actuating apparatus may be operative in a changing fashion in dependence on the operational data and measurements made by the measurement apparatus. The actuating apparatus may be operative in dependence on an actuating signal received by the actuating apparatus, such as an actuating signal generated by the processor. The actuating signal may be generated by the processor in dependence on at least one of the operational data and measurements made by the measurement apparatus. The actuating apparatus may be operative to change at least one of: duty cycle of the actuating signal; pulse width of the actuating signal; amplitude of the actuating signal; and profile of the actuating signal within a duty cycle.

The apparatus for topical application of material may comprise data storage. The apparatus for topical application of material may comprise at least one of a user operable control and a data communication port. The data communication port may provide for at least one of wireless communication, such as in accordance with the WiFi standard, and wired communication, such as by way of a USB port. The user operable control may be configured for user operation whereby operation of the apparatus for topical application of material may be controlled by the user. The apparatus for topical application of material may be configured such that data may be received thereby by way of the data communication port from further apparatus, such as the remote data store. The received data may be stored in the data storage. Data stored on the further apparatus may thus be uploaded to the apparatus for topical application of material. Further to operational data, the data stored on the further apparatus may comprise at least one of: data from previous operation of the apparatus for topical application of material in respect of: previous measurement by the measuring apparatus; previous characteristics of operation of the actuating apparatus; previous characteristics of operation of the application apparatus. Alternatively or in addition the data stored on the further apparatus may comprise phenotype data such as gender, age and ethnic origin. Data on skin characteristics of plural different classes of subject such as in respect of age group and/or recognised skin type may be stored. Such data on skin characteristics may be informed by the operational data. A user of the apparatus may select data for one of the plural classes which most closely matches her characteristics. Alternatively or in addition the data stored on the further apparatus may comprise data operative to provide for operation of at least one of the actuating apparatus and the application apparatus in a particular fashion, such as in respect of a subject belonging to a particular phenotype. Alternatively or in addition the data stored on the further apparatus may comprise skin property data such as skin impedance data.

The data storage may be operative to store data which provides for control of the apparatus for topical application of material in a predetermined fashion. Control may be in respect of at least one of the actuating apparatus and the application apparatus. The data may be configured to effect control to take account of at least one predetermined condition, for example a subject belonging to a particular group such as the subject belonging to a particular phenotype. The application apparatus may, for example, apply a certain amount per se of material for cosmetic purposes or apply material for cosmetic purposes at a particular rate in dependence on the data. Data may be stored in the data storage in dependence on operation of at least one of a user operable control and a data communication port comprised in the apparatus for topical application of material.

The apparatus for topical application of material may be configured to store, such as in data storage, data relating to operation of at least one of the measurement apparatus, the actuating apparatus and the application apparatus. The apparatus for topical application of material may thus be operative to store a profile of operation of apparatus for topical application of material. The apparatus for topical application of material may be configured to take such stored data into account during subsequent operation of the apparatus for topical application of material. Where the apparatus for topical application of material comprises a data communication port, the apparatus for topical application of material may be configured to provide for transmission of stored data from the apparatus for topical application of material by way of the data communication port to further apparatus, such as a remote data store.

The apparatus for topical application of material for cosmetic or medical purposes may be hand held and more specifically may be configured to be gripped in one hand. The apparatus for topical application of material for cosmetic or medical purposes may therefore comprise a housing, an exterior of the housing being configured to be gripped in one hand. The housing may support and more specifically may contain the measurement apparatus, the actuating apparatus, the application apparatus and the processor.

According to a third aspect of the present invention there is provided a method of topically applying material for cosmetic or medical purposes by way of apparatus for topical application of material, the method comprising:
measuring a property of skin of a human or animal subject;
changing a property of the skin of the human or animal subject;
applying material for cosmetic or medical purposes to the skin;
receiving operational data based on data received from each of plural other apparatus for topical application of material, said received data pertaining to the operation of the other apparatus for topical application of material; and
controlling the apparatus for topical application of material in dependence on the property measured by the measurement apparatus and the operational data.

Embodiments of the third aspect of the present invention may comprise one or more features of the first or second aspect of the present invention.

The present inventors have appreciated the feature of forming a profile for managing topical application of material for cosmetic purposes to be of wider applicability than hitherto described. Therefore and according to a fourth aspect of the present invention there is provided an arrangement for managing topical application of material for cosmetic purposes, the arrangement comprising:
plural measurement apparatus each configured to measure a property of skin of a respective human or animal subject;
a processor at a location remote from the plural measurement apparatus which is operative: to receive measurements from the plural measurement apparatus; and to form a profile in dependence on the measurements received from the plural measurement apparatus, the profile being for managing topical application of material for cosmetic purposes.

The arrangement for managing topical application of material for cosmetic purposes comprises plural measurement apparatus which are each configured to measure a property of skin of a respective human or animal subject. The arrangement further comprises a processor at a location remote from the plural measurement apparatus. The processor is operative to receive measurements from the plural measurement apparatus and to form a profile in dependence on the measurements received from the plural measurement apparatus. The profile is for managing topical application of material for cosmetic purposes.

The arrangement for managing topical application of material may further comprise plural apparatus for applying a cosmetic to the skin of the human or animal subject, the measurement apparatus being comprised in each of the plural apparatus. The apparatus for applying a cosmetic may comprise at least one of actuating apparatus and application apparatus. At least one of the actuating apparatus and the application apparatus may be operative in dependence on the profile received from the processor.

The profile formed by the processor may be modified in dependence on further measurements received from the plural measurement apparatus. The profile may thus be refined or modified on an ongoing basis. The profile may be formed in respect of a predetermined category of human or animal subject. The processor may store plural different profiles. One of the plural profiles may be selected and transmitted to apparatus for applying a cosmetic in accordance with a characteristic of the human or animal subject with which the apparatus for applying a cosmetic is to be or is being used.

Further embodiments of the fourth aspect of the present invention may comprise one or more features of any other aspect of the present invention.

According to a fifth aspect of the present invention there is provided apparatus for topical application of material for cosmetic or medical purposes, the apparatus comprising:
actuating apparatus configured to change a property of the skin of the human or animal subject;
application apparatus configured to apply material for cosmetic or medical purposes to the skin; and
a processor which is configured: to receive operational data based on data received from each of plural other apparatus for topical application of material, said received data pertaining to the operation of the other apparatus for topical application of material; and to control the apparatus for topical application of material in dependence on the operational data.

The apparatus for topical application of material may comprise a user interface, the apparatus for topical application of material being configured to inform the user by way of the user interface in dependence on the operational data. As described above, the user interface may comprise a display.

Further embodiments of the fifth aspect of the present invention may comprise one or more features of any other aspect of the present invention.

According to a further aspect of the present invention there is provided apparatus for topical application of material for cosmetic or medical purposes, the apparatus comprising: measurement apparatus configured to measure a property of skin of a human or animal subject; actuating apparatus configured to change a property of the skin of the human or animal subject; application apparatus configured to apply material for cosmetic or medical purposes to the skin; and a processor which is configured to control the actuating apparatus in dependence on the property measured by the measurement apparatus. Embodiments of the further aspect of the present invention may comprise one or more features of any other aspect of the present invention.

### BRIEF DESCRIPTION OF DRAWINGS

Further features and advantages of the present invention will become apparent from the following specific description, which is given by way of example only and with reference to the accompanying drawings, in which:

FIG. 1 is a block diagram representation of an arrangement for topical application of material for cosmetic or medical purposes according to the invention;

FIG. 2 is a schematic of apparatus for topical application of material according;

FIG. 3 is a block diagram of the main operative components of the apparatus of FIG. 2;

FIG. 4 shows an electrode assembly comprised in the apparatus of FIG. 2;

FIG. 5 is a flow chart showing the main steps during operation of the apparatus of FIG. 2 and the arrangement of FIG. 1;

FIG. 6 is a graph of skin impedance against rate of transepidermal water loss;

FIG. 7A is an example of an electrical equivalent model of the stratum corneum; and

FIG. 7B is a representative Cole-Cole plot for skin tissue.

### DESCRIPTION OF EMBODIMENTS

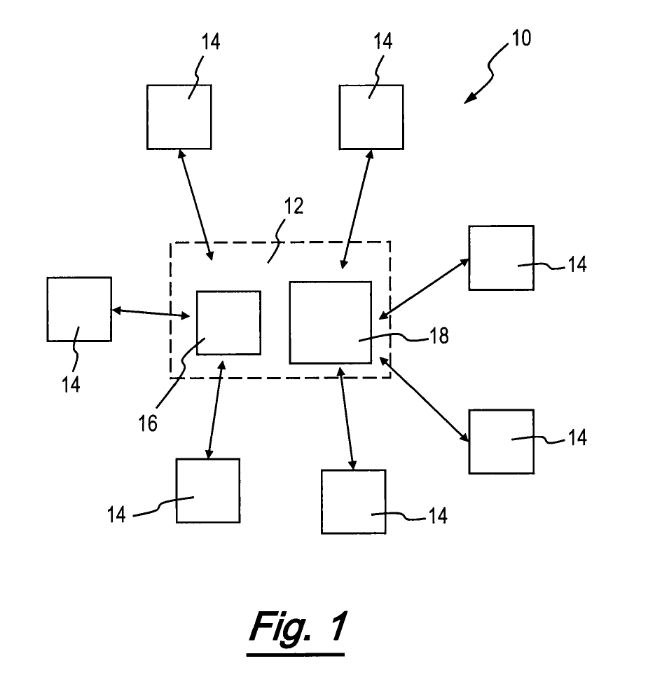

A block diagram representation of an arrangement for topical application of material for cosmetic or medical purposes 10 is shown in FIG. 1. The arrangement for topical application of material 10 comprises a cloud based arrangement 12 and plural apparatus for topical application of material for cosmetic or medical purposes 14. The cloud based arrangement 12 comprises a central processor 16 and a data store 18. The cloud based arrangement 12 is of conventional form and function. The plural apparatus for topical application of material are of the same form and function as each other. The plural apparatus for topical application of material 14 are remote from the cloud based arrangement 12 and remote from each other and may be located in different countries. Typically apparatus for topical application of material 14 is located in the home of the user.

Each apparatus for topical application of material 14 is in communication with the cloud based arrangement 12 by way of a wired or wireless link to a local communications hub (not shown) which is then communication with the cloud based arrangement 12 by way of the Internet. Where communication between apparatus for topical application of material 14 and the local communications hub is wired, a wired link is established by way of a USB socket on each of the apparatus for topical application of material 14 and the local communications hub. Where communication between apparatus for topical application of material 14 and the local communications hub is wireless, the apparatus for topical application of material 14 and the local communications hub are configured for communication in accordance with either the WiFi standard or the Bluetooth standard. The local communications hub typically is a home or small office router of conventional form and function. In practice, the arrangement for topical application of material for cosmetic or medical purposes 10 comprises many more apparatus for topical application of material 14 than are shown in FIG. 1. As will become apparent from the following description, utility improves with the number of apparatus for topical application of material 14 comprised in the arrangement 10. The apparatus for topical application of material 14 is described further below.

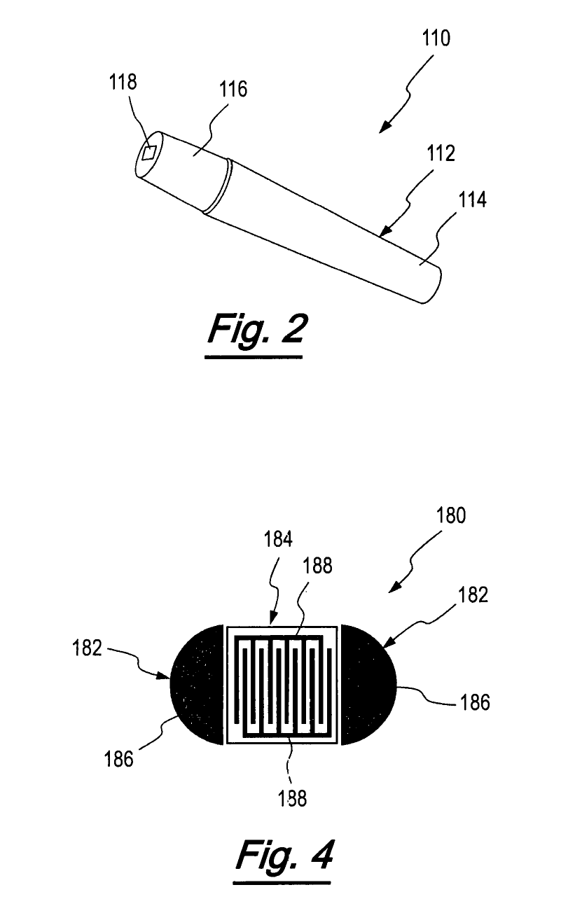

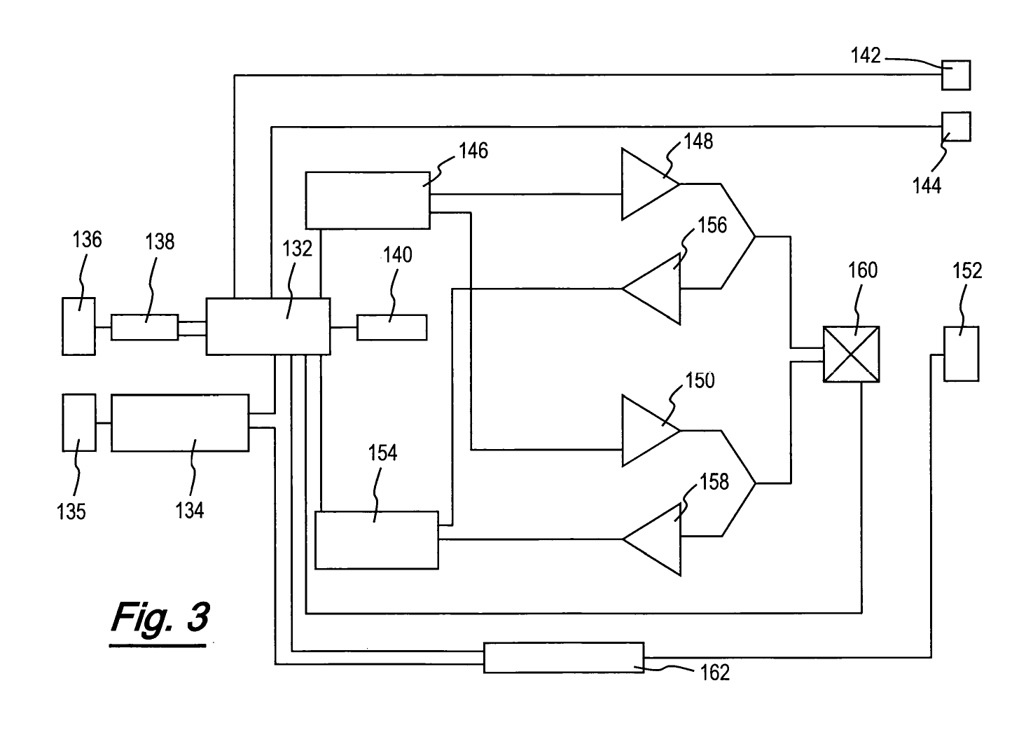

A schematic of apparatus for topical application of material 110 is shown in FIG. 2. The apparatus comprises a housing 112 which supports and contains components of the apparatus and defines an external surface of the apparatus. The housing comprises two parts: a main body 114; and an operative body 116. The housing is of a shape and size that the main body can be gripped in the hand of a user. The main body 114 contains and supports a reservoir (not shown) which contains a cosmetic agent such as skin-care lotion (which constitutes material for cosmetic purposes) or a medical agent (which constitutes material for medical purposes). The main body 114 also contains and supports six rechargeable AA NiMH battery cells which provide electrical power for the apparatus 110. The battery cells are rechargeable by way of an external battery charger in accordance with conventional practice. The operative body 116 comprises electronic and electro-mechanical components of the apparatus 110. The electronic and electro-mechanical components are described below with reference to FIG. 3. The distal end of the operative body 116 defines a substantially planar surface on which an electrode assembly 118 is mounted. The electrode assembly 118 is described below with reference to FIG. 3 and subsequently in more detail with reference to FIG. 4.

A block diagram of the main operative components of the apparatus of FIG. 2 is shown in FIG. 3. The main operative components comprise an ARM Cortex M3 embedded processor 132, electrical power supply circuitry 134, a USB communication port 136, communication port driver circuitry 138, a loudspeaker 140, LED indicators 142 and an on-off switch 144. The ARM Cortex M3 embedded processor 132 is operative to control operation of the apparatus 110 of FIG. 2 in respect of measurement, actuation and application of cosmetic or medical agent as is described further below. The ARM Cortex M3 embedded processor 132 is therefore operative to store in integral memory firmware to control such operation and to provide for control and operation as otherwise described herein. The provision of such firmware is within the ordinary design capabilities of the notionally skilled person. The ARM Cortex M3 embedded processor 132 comprises integral components such as timers, an analogue-to-digital converter, and a digital-to-analogue converter and plural digital input/output lines. The analogue-to-digital converter is operative to provide for analogue-to-digital conversion of acquired analogue signals for processing within the ARM Cortex M3 embedded processor 132. The digital-to-analogue converter is operative to convert digital data to analogue signals for the actuating apparatus described below. The digital input/output lines are operative to control components of the apparatus 110, such as in respect of putting electronic components into a tri-state condition or into or out of a power conserving mode, and to control whatever further external apparatus may be provided, such as skin abrading apparatus.

The electrical power supply circuitry 134 comprises the electric batteries described above with reference to FIG. 2, and is otherwise configured by way of bandgap reference circuitry and voltage generation and regulation circuitry to provide electrical power rails for the electronic circuitry and required reference voltages. The electric batteries are recharged when recharging current is provided by way of gold plated copper terminals 135 provided on the exterior of the housing 112. The design of electrical power supply circuitry 134 is within the ordinary design capabilities of the notionally skilled person. The loudspeaker 140 is operative under control of the ARM Cortex M3 embedded processor 132 to provide audible notification as to when components of the apparatus 110 of FIG. 2, such as the measurement apparatus and the actuating apparatus, are operating. The LED indicators 142 are operative under control of the ARM Cortex M3 embedded processor 132 to provide visible notification as to when the apparatus 110 of FIG. 2 is switched on and otherwise, such as by use of different colours, to provide visible notification as to when different parts of the apparatus 110 of FIG. 2 are operating, such as when each of the measurement apparatus and the actuating apparatus is operating. The communication port driver circuitry 138, which is under control of the ARM Cortex M3 embedded processor 132, drives the USB communication port 136 to provide for reception of data by and transmission of data from the apparatus 110 of FIG. 2 as is described in more detail below. In alternative forms, the communication port driver circuitry 138 is operative to drive a WiFi or Bluetooth transceiver to provide for wireless communication of data to and from the apparatus. Design of the apparatus to provide for WiFi or Bluetooth communication will be a matter of ordinary design for the skilled reader. The apparatus 110 of FIG. 2 is brought into data communication with a computer, such as a laptop, by way of a USB cable which is connected to the USB communication port 136 or with a home or small office router by way of a WiFi link. Although not shown in FIG. 2, the apparatus 110 comprises a temperature sensor and a humidity sensor which are operative to measure the temperature and humidity respectively of the environment around the apparatus 110. Although not shown in FIG. 2, the apparatus 110 comprises a GPS receiver which is operative to determine the location of the apparatus 110.

The main operative components of FIG. 3 further comprise a signal generator 146, actuation signal conversion and conditioning circuitry 148, measurement signal conversion and conditioning circuitry 150 and the electrode assembly 118, 152. The signal generator 146 is under control of and receives data from the ARM Cortex M3 embedded processor 132 and is operative to drive each of the actuation signal conversion and conditioning circuitry 148 and measurement signal conversion and conditioning circuitry 150 at different times. The signal generator 146 is operative to determine voltage and current amplitude, frequency, duty cycle and duration of signals generated. The actuation signal conversion and conditioning circuitry 148 comprises a 12-bit digital-to-analogue converter from Analog Devices, namely an AD5339, and other circuitry to provide for current limit control and voltage compliance control depending on the mode of operation and to interface with the electrode arrangement 152. The measurement signal conversion and conditioning circuitry 150 comprises a digital sine wave generator, namely an AD9832 from Analog Devices, a 25 MHz master clock generator, namely a SG8002DCOHB from Epson, and other circuitry to interface with the electrode arrangement 152.

The main operative components of FIG. 3 yet further comprise a signal measurement arrangement 154, a first measurement interface arrangement 156, and a second measurement interface arrangement 158. The signal measurement arrangement 154 comprises an 18-bit analogue-to-digital converter, namely an MCP3421 from Microchip, and is under control of and provides digital data to the ARM Cortex M3 embedded processor 132 in dependence on analogue signals received from the first measurement interface arrangement 156 and the second measurement interface arrangement 158 at different times. The signal measurement arrangement 154 is operative to determine voltage amplitude and phase angle and current amplitude and phase angle. The first measurement interface arrangement 156 comprises circuitry to interface with the electrode arrangement 152. The second measurement interface arrangement 158 comprises circuitry to interface with the electrode assembly 152. In addition the main operative components of FIG. 3 comprise an electrode assembly switching control 160 and a pump arrangement 162 (which constitutes application apparatus). The electrode assembly switching control 160 is under the control of the ARM Cortex M3 embedded processor 132 and is operative to determine the mode of operation of the electrode assembly 152 as is described further below. The pump arrangement 162 is under the control of the ARM Cortex M3 embedded processor 132 and is operative to pump cosmetic or medical agent contained in the reservoir of the apparatus 110 of FIG. 2. A conduit from the reservoir is operative to convey pumped cosmetic or medical agent to an aperture beside the electrode assembly 152 whereby cosmetic or medical agent is dispensed from the apparatus 110 of FIG. 2.

The electrode assembly 118, 152 of FIGS. 2 and 3 is shown in more detail in FIG. 4. The electrode assembly 180 of FIG. 4 comprises a first electrode arrangement 182 and a second electrode arrangement 184. The first electrode arrangement 182 comprises two first electrodes 186 which are spaced apart from each other such that the second electrode arrangement 184 is disposed therebetween. The two first electrodes 186 lie in substantially the same plane, each define a semicircle in area and are disposed such that the linear bases of the semicircles face each other. The two first electrodes 186 are formed of gold plated copper and are not covered by an insulating material. The second electrode arrangement 184 comprises two second electrodes 188 which each define serpentine paths and are disposed relative to each other such that they define an interdigitated structure. The two second electrodes 188 lie in substantially the same plane and in the same plane as the two first electrodes 186. The two second electrodes 188 are formed of gold plated copper. A layer of an insulating material, such as polyurethane, covers the two second electrodes 188. The width of each of the two second electrodes 188 is 0.1 mm and the separation between the two second electrodes 188 is 0.06 mm. Different electrode spacings are used depending on the thickness of the skin to which the cosmetic or medical agent is to be applied. An electrode spacing of 0.06 mm is appropriate where the stratum corneum is thin, such as around the eye. An electrode spacing nearer to 0.1 mm is appropriate where the stratum corneum is thicker, such as the heel. The total area of the electrode assembly 180 is 2 cm² (2 cm long by 1 cm wide).

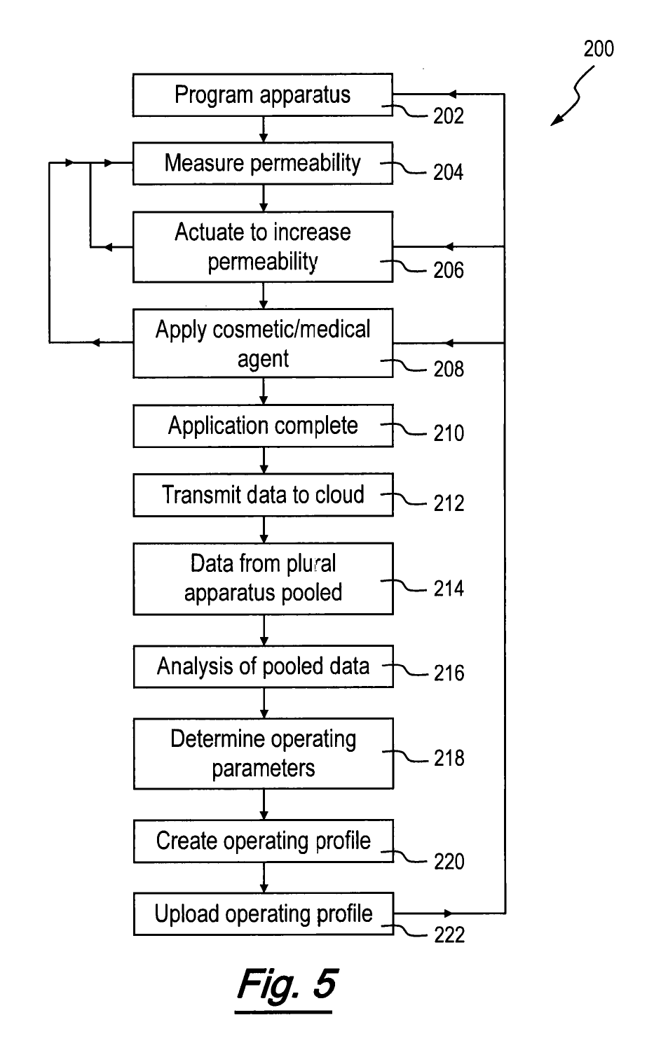

Operation of the arrangement 10 and the apparatus 14, 110 described above with reference to FIGS. 1 to 4 will now be described with reference to the flow chart 200 shown in FIG. 5. After the apparatus 14, 110 of FIGS. 1 and 2 is switched on by way of the on/off switch 144 the apparatus is programmed 202. Programming the apparatus comprises configuring the apparatus to carry out a skin preparation and cosmetic or medical agent application regime. The apparatus is configured by downloading configuration data either from a computer which stores the configuration data by way of a USB cable to the USB communication port 36 or from the cloud based arrangement 12 by way of the Internet and the local WiFi link. The downloaded configuration data is then conveyed to the ARM Cortex M3 embedded processor 132 for storage therein. The configuration data comprises phenotype data such as gender, age and ethnic origin and also skin type data such as normal, dry, oily and combination. The user makes an appropriate selection on the computer before the configuration data is downloaded from the computer or on the apparatus 14, 110 before the configuration data is downloaded from the cloud based arrangement 12. The configuration data also comprises historic data which has been uploaded from previous use of the apparatus 14, 110 of FIGS. 1 and 2, with such historic data being operative to provide for refining of the skin preparation and cosmetic or medical agent application regime. By way of example, frequency of use of the apparatus and the amount of cosmetic or medical agent dispensed on each occasion is stored. The processor 132 is operative in dependence on the stored data to restrict the amount of cosmetic or medical agent dispensed during further use of the apparatus. For example a maximum weekly dose for a particular cosmetic or medical agent may be 2 ml based on previous use. The processor 132 is therefore operative to provide that no more than this amount is dispensed in a seven day period or to reduce the amount dispensed during the latter part of the seven day period. If a user does not use the apparatus for one or more days during a seven day period the processor is operative to increase the amount dispensed during the remaining days of the seven day period.

The second stage comprises measurement of the permeability of the skin 204 during which the electrode assembly 118 is brought into contact with the skin. Two forms of measurement are made: skin impedance measurement; and skin capacitance measurement. Each form of measurement will now be described in turn although the two forms of measurement are both used to provide enhanced characterisation of skin permeability.

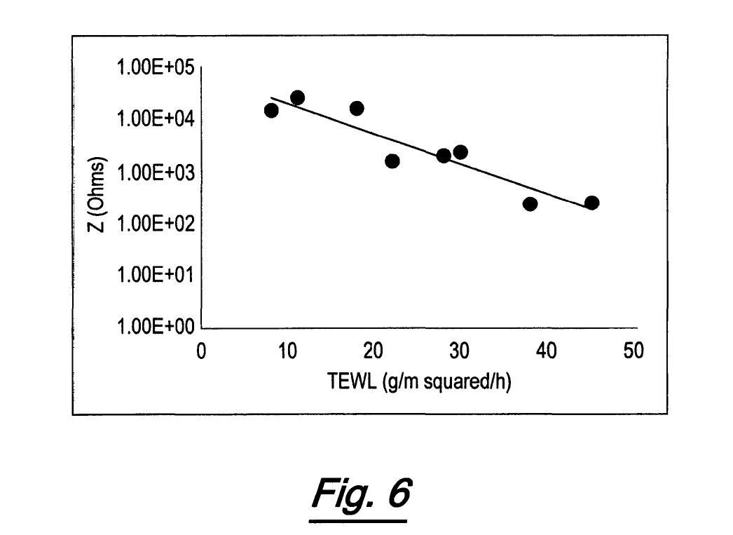

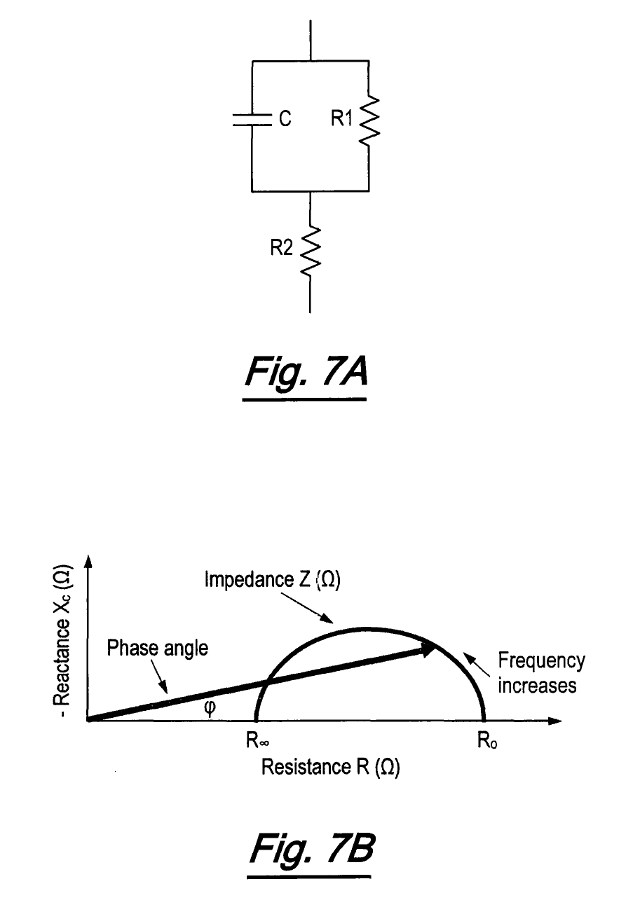

Considering skin impedance measurement first, the signal generator 146, the measurement signal conversion and conditioning circuitry 150 and the first electrode arrangement 182 are operative together under control of the ARM Cortex M3 embedded processor 132 to apply an alternating current signal of swept frequency between 100 Hz and 1 MHz or an alternating current signal comprising frequencies between 100 Hz and 1 MHz. The signal measurement arrangement 154, the second measurement interface arrangement 158 and the first electrode arrangement 182 are operative together under control of the ARM Cortex M3 embedded processor 132 to measure the current signal between the electrodes of the first electrode arrangement 182 at different frequencies. The ARM Cortex M3 embedded processor 132 is operative to determine the impedance on the basis of the current measurements and the applied voltage and then to determine the real and imaginary parts of the impedance. The thus determined real part of the impedance is then correlated with skin permeability by the ARM Cortex M3 embedded processor 132 in dependence on a model stored in the processor 132. A graph which relates skin impedance to transepidermal water loss is shown in FIG. 6. The graph of FIG. 6 forms the basis for a model that relates impedance to skin permeability. The graph of FIG. 6 and the model based thereon are described further below. The ARM Cortex M3 embedded processor 132 is further operative to determine a capacitance of the skin in dependence on the imaginary part of the impedance, the frequency of the applied signal and a resistance value representing resistance of an outermost layer of the stratum corneum in accordance with the model of the skin described below with reference to FIGS. 7A and 7B.

Turning now to consider skin capacitance measurement, the signal generator 146, measurement signal conversion and conditioning circuitry 150 and the second electrode arrangement 184 are operative together under control of the ARM Cortex M3 embedded processor 132 to apply an electric field to the skin. More specifically the electrodes of the second electrode arrangement 184 are operative as plate of a capacitor with the skin constituting the dielectric between the plates of the capacitor. The signal generator 146 is operative to provide for application of a signal of alternating current form to the electrodes of the second electrode arrangement 184. The signal measurement arrangement 154 and the second measurement interface arrangement 158 are operative together under control of the ARM Cortex M3 embedded processor 132 to provide for measurement of the current and voltage between the electrodes of the second electrode arrangement 184. The ARM Cortex M3 embedded processor 132 is operative to determine the capacitive reactance and then relative permittivity in dependence thereon. The ARM Cortex M3 embedded processor 132 is also operative to provide for determination the thickness of the skin. Determination of relative permittivity and of skin thickness will now be described further below.

The reactance of the capacitor defined by the electrode plates of the second electrode arrangement 184 and the skin is given by:

$${X}_{C} = \frac{1}{2 π fC}$$

Re-arranging we obtain:

$$C = \frac{1}{2 π {fX}_{c}}$$

The capacitance is related to the permittivity of the material between the plates by:

$$C = ɛ \frac{A}{d}$$

Combining the two immediately preceding equations and making ε, the relative permittivity, the subject of the combined equation we obtain:

$$ɛ = \frac{d}{2 π {fX}_{c} A}$$

A, the area of the plates and f, the frequency, are known. As mentioned above, the capacitive reactance, X_{c}, is measured and thus known. The distance separating the plates, d, is estimated based on the typical thickness of the part of the skin being measured. The relative permittivity, ε, is therefore calculated.

As described above, measurement using the first electrode arrangement 182 also provides for determination of the capacitance C. In view of capacitance being related to relative permittivity by:

$$C = ɛ \frac{A}{d}$$

We re-arrange to obtain:

$$d = ɛ \frac{A}{C}$$

The capacitance, C, is provided by measurement with the first electrode arrangement 182, the relative permittivity, ε, is provided by measurement with the second electrode arrangement 184 and the area, A, of the second electrode arrangement is known. Hence d, the plate separation or more specifically the depth of the stratum corneum, is calculated to thereby provide a more accurate value for d.

Returning to FIG. 5 now that the permeability of the skin has been determined, the ARM Cortex M3 embedded processor 132 is operative to determine whether or not the permeability meets the requirements stored at the first stage 202 of the process. If not, the process progresses to a step in which the permeability of the skin is increased 206. The permeability of the skin is increased by two approaches: application of an electric potential signal; and application of an electric current signal. Each approach will now be described in turn although the two approaches are both used to provide for an enhanced increase in permeability of the skin.

Considering application of an electric potential signal first, the signal generator 146, the actuation signal conversion and conditioning circuitry 148 and the second electrode arrangement 184 are operative together under control of the ARM Cortex M3 embedded processor 132 to apply an electric potential in the range of 20 volts to 140 volts in the form of a pulse of duration of 10 μs to 10 ms. The pulse is operative to reversibly form plural pathways through the stratum corneum to thereby increase permeability. During application of the electric potential, the signal measurement arrangement 154 and the first measurement interface arrangement 156 are operative to measure the electric potential at the second electrode arrangement 184 to monitor the applied electric potential and provide feedback control.

Turning now to consider the application of an electric current signal, the signal generator 146, the actuation signal conversion and conditioning circuitry 148 and the first electrode arrangement 182 are operative together under control of the ARM Cortex M3 embedded processor 132 to apply a substantially constant current to the skin by way of the electrodes of the first electrode arrangement 182. A potential of no more than 50 Volts and a current of up to 500 μA/cm² are applied to the skin. During application of the current, the signal measurement arrangement 154 and the first measurement interface arrangement 156 are operative to measure the current at the first electrode arrangement 182 to monitor the applied current and provide feedback control. Application of the current is operative to maintain the pathways formed by the pulsed electric potential in the absence of the latter.

After step 206 of FIG. 5, the apparatus 14, 110 of FIGS. 1 and 2 is operative to measure the permeability as described above with reference to step 204 of FIG. 5. Steps 206 and 204 are repeated until a desired permeability is achieved.

When a desired permeability is achieved, operation of the apparatus 14, 110 of FIGS. 1 and 2 progresses to application of the cosmetic or medical agent to the skin 208. More specifically the pump arrangement 162 is operative under control of the ARM Cortex M3 embedded processor 132 to pump cosmetic or medical agent from the reservoir through the aperture provided adjacent the electrode assembly 118 onto the skin. The amount and rate of application of cosmetic or medical agent to the skin is determined in accordance with data stored in the ARM Cortex M3 embedded processor 132 at step 202 in FIG. 5 to meet a desired cosmetic or medical agent application regime. In certain forms, application of cosmetic or medical agent takes place in plural stages with measurement performed in accordance with step 204 and actuation in accordance with step 206 performed between stages of application of cosmetic or medical agent.

When application of cosmetic or medical agent is complete 210, data relating to the just completed skin preparation and cosmetic or medical agent application process is stored by the ARM Cortex M3 embedded processor 132. The stored data is either transmitted to the user's computer or to the cloud based arrangement 12 for use as historic data in subsequent treatments as described above. If the stored data is transmitted to the user's computer it is also transmitted to the cloud based arrangement 12, 212 for use other than as historic data as will now be described. The stored data received by the cloud based arrangement 12 also comprises temperature and humidity data from measurements made by the temperature and humidity sensors and location data from the GPS receiver. The stored data received by the cloud based arrangement 12 is pooled under control of the central processor 16, 214 with stored data received from each of the other apparatus for topical application of material 14 of FIG. 1. The cloud based arrangement 12 thus comprises a database in the data store 18 of data which relates to completed skin preparation and cosmetic or medical agent application processes from various apparatus for topical application of material 14. The database of data is updated on an ongoing basis as further use is made of the various apparatus for topical application of material 14. The database thus contains data which characterises use of apparatus for topical application of material 14 with a wide variety of subjects and in respect of differing circumstances, such as different times of the day, different dates, different temperatures, different humidity, different locations, etc. Such a database is employed to provide for improved use of each apparatus for topical application of material 14 as will now be described.

The central processor 16 is operative to analyse the data contained in the database on an ongoing basis 216 and to draw conclusions in dependence on the analysis. Thereafter operating parameters are determined in dependence on the conclusions drawn 218. In some circumstances the central processor 16 is operative in respect of these steps without user input. In other circumstances the central processor 16 is operative in respect of these steps with user input. In such other circumstances, user input is, for example, involved in drawing conclusions after analysis of the data. After operating parameters have been determined, the operating parameters are stored in the data store 18 as an operating profile 220 comprising data fields which identify appropriate circumstances for use of the operating parameters such as with a particular demographic, for example Italian women between the ages of 40 and 49. Plural different operating profiles are created over time. Upon subsequent operation of apparatus for topical application of material 14 which is configured for use with a particular demographic, the apparatus for topical application of material 14 is operative to upload the operating profile in question 222. Depending on circumstances of intended use, the apparatus for topical application of material 14 is configured to at least one of: modify initial programming of the apparatus 14, 202 by changing the like of target values for actuation and application; modify operation of at least one of the actuating apparatus 206 and the application apparatus 208 in dependence of the uploaded operating profile. Operation of each of the plural apparatus for topical application of material 14 is thus influenced by the data collected by the cloud based arrangement 12 from all of the plural apparatus for topical application of material 14 to provide for improved treatment. Furthermore collection of data over time from all of the plural apparatus for topical application of material 14 provides for improved modelling of usage of apparatus for topical application of material. It is noted that as the number of plural apparatus for topical application of material 14 increases there is an improved basis for modelling.

The central processor 16 is operative to analyse the collected data for several purposes. A first purpose is to identify factors common to plural instances of use of apparatus for topical application of material 14. For example, analysis reveals that skin measurements are affected in a consistent fashion by change in temperature or change in humidity as reflected by the temperature and humidity data. By way of further example, analysis reveals that a skin treatment regime should be changed to take account of climate as inferred from location. As a result of such an outcome, the central processor 16 is operative to determine how target values for actuation and application are to be changed upon initial programming of the apparatus 14, 202 to take account of local change in temperature or humidity or location as determined by the GPS receiver.

A second purpose is optimising treatment with apparatus for topical application of material 14. Data received from various apparatus for topical application of material 14 is analysed under control of the central processor 16 to determine an optimum skin stimulation regime. For example a first cluster of data may relate to use of a particular product with a stimulation pattern involving low amplitude and high frequency and second cluster of data may relate to use of the particular product with a stimulation pattern involving high amplitude and low frequency. Analysis may, for example, reveal that the first cluster of data involves more effective application of the particular product than the second cluster of data. The central processor is then operative to determine how operating parameters for the actuating apparatus should be set such that they are in accordance with operating parameters for the first cluster of data. The operating parameters comprise: duty cycle of the actuating signal; pulse width of the actuating signal applied by the actuating apparatus; amplitude of the actuating signal; profile of the actuating signal within a duty cycle; amount of material dispensed on the skin; rate of material dispensed on the skin; and consistency of material dispensed on the skin.

A third purpose is arriving quickly at an effective treatment regime for a new cosmetic or medical agent. When the new cosmetic or medical agent is used for the first time in various apparatus for topical application of material 14, the data from various apparatus is collected in the cloud based arrangement 12. The thus collected data is analysed under control of the central processor 16 to determine an optimum treatment regime and corresponding operating parameters. Such corresponding operating parameters are then used as described above to modify control of at least one of the actuating apparatus and the application apparatus during subsequent use of apparatus for topical application of material with the new cosmetic or medical agent.

A fourth purpose is determining a fresh operating profile. Freshly collected data from apparatus for topical application of material 14 is compared with previously collected data to determine whether or not the freshly collected data is in conformance with one of previously determining operating profiles. If the freshly collected data is not in conformance with one of previously determining operating profiles, the central processor 18 is operative to form a fresh operating profile appropriate to the freshly collected data. The fresh operating profile is then used as described above to control at least one of the actuating apparatus and the application apparatus during subsequent use of apparatus for topical application of material to provide a more appropriate treatment regime.

A fifth purpose is informing the user of the apparatus for topical application of material 14. The loudspeaker 140 under control of the ARM Cortex M3 embedded processor 132 provides a message to the user in dependence on the operating profile and measurements made by the measurement apparatus. By way of example, the user is informed by way of the loudspeaker 140 that the formulation of the cosmetic material should be changed. In another form of this example, a replacement cartridge containing the changed formulation is sent to the user and without user input with the user being informed accordingly or not by way of the loudspeaker 140 depending on configuration of the apparatus 14. The user is thus provided with a customised cosmetic formulation on an ongoing basis. By way of another example, the user is informed by way of the loudspeaker 140 regarding one or more of timing and frequency of use of the apparatus, volume of cosmetic material to be applied or rate of application of cosmetic material. Instead of providing a message by way of the loudspeaker 140, a communication link is established between the apparatus for topical application of material 14 and a Personal Computer (PC) by way of the USB communication port 136 and information for the user is conveyed to the PC by way of the communication link for output to the user by way of the PC.

FIG. 6 shows a graph which relates skin impedance to the rate of transepidermal water loss (TEWL). Transepidermal water loss is a measurement of water lost through the skin by way of the stratum corneum other than by sweating. As per FIG. 6, the units of water loss are expressed in g/m²/h (grams per meter squared per hour). As can be seen from FIG. 6, there is a generally linear relationship between the log of skin impedance and the rate of transepidermal water loss with impedance decreasing as the rate of transepidermal water loss increases. It is known that the rate of transepidermal water loss corresponds to skin permeability. The model used by the ARM Cortex M3 embedded processor 132 in determining skin permeability is based on the relationship of FIG. 6 between skin impedance and the rate of transepidermal water loss.

A simple electrical equivalent model of the stratum corneum is shown in FIG. 7A. The model consists of a first resistor R₁ in parallel with a capacitor C with this parallel arrangement being in series with a second resistor R₂. C and R₁ represent the capacitance and resistance of the outermost layer of the stratum corneum and R₂ represents the resistance found in deeper layers of the stratum corneum. Typical values for R₁ range from 100Ω to 5 MΩ cm² and R₂ from 0.1Ω to 1 kΩ cm². The model reflects the frequency dependence of skin and in particular the decrease in magnitude of the impedance as frequency increases. Based on skin impedance measurements as described above and the theory for biological tissues according to the Cole brothers (Bioelectrical impedance analysis—part I: review of principles and methods, Kyle, U. G., I. Bosaeus, et al. (2004), Clinical Nutrition 23(5): 1226-1243), values for R₁, R₂ and C can be calculated. A typical complex plot obtained when impedance of any biological tissue is analysed is shown in FIG. 7B. According to the Cole brothers' empirical equation the impedance is given by:

$$Z = {R}_{∞} + \frac{{R}_{0} - {R}_{∞}}{1 + {( j ω τ )}^{α}} \qquad Equation 1$$

Where R_{∞} is the resistance at very high frequencies where the semicircle of FIG. 6 crosses the x axis (i.e. when the reactance is zero), R₀ is the resistance at very low frequencies where the semicircle of FIG. 7B crosses the x axis (i.e. when the reactance is zero), ω is the angular frequency, τ is a time constant and φ=α(π/2) is the constant phase angle. The general equation for this circuit can be written as:

Z=Zₛₑᵣᵢₑₛ+Zₚₐᵣₐₗₗₑₗ Equation 2

Based on a comparison of Equation 1 with Equation 2 the following equations can be obtained:

$$\begin{matrix}{Z}_{series} = {R}_{∞} = {R}_{2} & Equation 3 \\ {Z}_{parallel} = \frac{{R}_{0} - {R}_{∞}}{1 + {( j ω τ )}^{α}} = \frac{{R}_{1}}{1 + j ω {R}_{1} C} & Equation 4\end{matrix}$$

The parallel impedance has real and imaginary parts and can be represented in the Cartesian form as:

$${Z}_{parallel} = {Z}^{′} + {Z}^{″} = \frac{{R}_{1}}{1 + {ω}^{2} {R}_{1}^{2} {C}^{2}} - j \frac{ω {R}_{1}^{2} C}{1 + {ω}^{2} {R}_{1}^{2} {C}^{2}} \qquad Equation 5$$

In Equation 5 Z″ is the part of interest which provides for calculation of the value of C:

$${Z}^{″} = j \frac{ω {R}_{1}^{2} C}{1 + {ω}^{2} {R}_{1}^{2} {C}^{2}} \qquad Equation 6$$

where Z″ is measured, ω is known and R₁ is R₀ at low frequencies or R_{∞} at high frequencies. Equation 6 is solved for C in view of Z″, R₁ and ω being known.

1. Apparatus for topical application of material for cosmetic or medical purposes, the apparatus comprising:
measurement apparatus configured to measure a property of skin of a human or animal subject;
actuating apparatus configured to change a property of the skin of the human or animal subject;
application apparatus configured to apply material for cosmetic or medical purposes to the skin; and
a processor which is configured: to receive operational data based on data received from each of plural other apparatus for topical application of material, said received operational data pertaining to operation of the other apparatus for topical application of material; and to control the apparatus for topical application of material in dependence on the property measured by the measurement apparatus and the operational data.

2. Apparatus according to claim 1, in which the operational data comprises at least one profile appropriate to a user of the apparatus for topical application of material.

3. Apparatus according to claim 1 configured such that at least one of the actuating apparatus and the application apparatus is controlled in dependence on the property measured by the measurement apparatus and the operational data.

4. Apparatus according to claim 1 further comprising a user interface, the apparatus for topical application of material being configured to inform a user by way of the user interface in dependence on the property measured by the measurement apparatus and the operational data.

5. Apparatus according to claim 4 in which the processor is configured to determine that a formulation of the material for cosmetic or medical purposes should be changed in dependence on the property measured by the measurement apparatus and the operational data and to inform the user accordingly by way of the user interface.

6. Apparatus according to claim 4 in which the processor is operative to make a determination concerning use of the apparatus for topical application of material in dependence on the property measured by the measurement apparatus and the operational data and to inform the user accordingly by way of the user interface.

7. Apparatus according to claim 1 further comprising data communication apparatus which is operative to receive the operational data from a remote data store and to transmit data pertaining to the operation of the apparatus for topical application of material to the remote data store.

8. Apparatus according to claim 7 in which the data communication apparatus is operative to transmit data relating to operation of at least one of the measurement apparatus, the actuating apparatus and the application apparatus.

9. Apparatus according to claim 8 in which the transmitted data comprises at least one of: measurements made with the measurement apparatus; characteristics of an actuating signal received by the actuating apparatus; and characteristics of an actuating signal received by the application apparatus.

10. Apparatus according to claim 7 in which the data communication apparatus is operative to transmit data relating to measurement made by way of at least one sensor comprised in the apparatus for topical application of material.

11. Apparatus according to claim 10 further comprising at least one of a temperature sensor and a humidity sensor, the transmitted data relating to measurement made by at least one of a temperature sensor and a humidity sensor.

12. Apparatus according to claim 1 configured to determine its location, to provide corresponding location data and to transmit the corresponding location data by way of data communication apparatus comprised in the apparatus for topical application of material, the received operational data being further based on location data from the other apparatus for topical application of material.

13. Apparatus according to claim 12 comprising a sensor and location determining apparatus, the processor being configured to control the apparatus for topical application of material in dependence on measurement data from the sensor and location data from the location determining apparatus.

14. Apparatus according to claim 13 in which the processor is operative: to make an inference regarding climate based on the location data and the measurement data; and to control the apparatus for topical application of material in dependence on operational data selected in dependence on the inference regarding climate.

15-18. (canceled)

19. Apparatus according to claim 1 configured to be gripped in one hand and operated when so gripped.

20. An arrangement for topical application of material for cosmetic or medical purposes, the arrangement comprising: plural apparatus for topical application of material for cosmetic or medical purposes, each according to claim 1; and a data store remote from the plural apparatus.

21. (canceled)

22. An arrangement according to claim 20 in which each of the plural apparatus for topical application of material is operative: to receive operational data from the data store; and to transmit data pertaining to the operation of the apparatus for topical application of material to the data store.

23. An arrangement according to claim 20 in which the operational data which the data store provides to apparatus for topical application of material is updated on an ongoing basis in dependence on fresh data received from the plural apparatus for topical application of material.

24. An arrangement according to claim 20 in which each of the plural apparatus for topical application of material is operative on the basis of first and second feedback loops, the first feedback loop being local to the apparatus for topical application of material and comprising the measurement apparatus comprised in the apparatus and at least one of the actuation apparatus and the application apparatus comprised in the apparatus, the second feedback loop being remote in part from the apparatus for topical application of material and comprising the plural apparatus for topical application of material, the data store and each of the plural apparatus for topical application of material.

25. An arrangement according to claim 24 in which the second feedback loop is operative to influence operation of the first feedback loop.

26-28. (canceled)
